(19) United States
(12) Patent Application Publication
(10) Pub. No.: US 2024/0245781 A1
(43) Pub. Date: Jul. 25, 2024

(54) USE OF DESTRUCTURIZED STARCH AS A THICKENING AGENT AND COMPOSITIONS CONTAINING IT

(71) Applicant: Novamont S.p.A., Novara (IT)

(72) Inventors: Paolo MAGISTRALI, Novara (IT); Ioanna PETRAKOU, Novara (IT); Alessandro MONTINO, Robbio (IT)

(73) Assignee: Novamont S.p.A., Novara (IT)

(21) Appl. No.: 18/626,374

(22) Filed: Apr. 4, 2024

Related U.S. Application Data
(63) Continuation of application No. 16/069,898, filed on Jul. 13, 2018.

(30) Foreign Application Priority Data
Jan. 19, 2016 (IT) 102016000004460

(51) Int. Cl. A61K 47/36; A61K 8/73

(52) U.S. Cl. CPC A61K 47/36; A61K 8/732; A61K 2800/48

(57) ABSTRACT

This invention relates to the use of destructurized starch as a thickening agent in cosmetic, dermatological and pharmaceutical compositions, paints, phytosanitary products and detergents.

### CROSS-REFERENCE TO RELATED APPLICATIONS

This application claims priority to and is a continuation of U.S. patent application Ser. No. 16/069,898, filed on Jul. 13, 2018, which is the National Phase filing under 35 U.S.C. § 371 of PCT/EP2017/051036 filed on Jan. 19, 2017; which application in turn claims priority to Application No. 102016000004460 filed in Italy on Jan. 19, 2016 under 35 U.S.C. § 119. The entire contents are hereby incorporated by reference.

### DESCRIPTION

This invention relates to the use of destructurized starch as a thickening agent in cosmetic, dermatological and pharmaceutical compositions, paints, phytosanitary products and detergents.

Thickening agents have long been used in various sectors of industry to regulate the viscosity of a wide range of products, such as for example cosmetic, dermatological and pharmaceutical compositions, paints, phytosanitary products and detergents. Many thickening agents are known in commerce, mainly all of synthetic origin.

However, despite showing good performance, these thickening agents of synthetic origin generally have a high production cost and, being made from raw materials of fossil origin contribute to prejudice reserves of natural resources for future generations.

Recently the possibility of replacing these thickening agents of synthetic origin with other thickening agents of renewable origin, typically based on polymers of natural origin, has been assessed with increasing interest in various sectors of industry.

For example, WO 2015/097181 describes cosmetic compositions in the form of oil-in-water emulsions incorporating a thickening agent comprising hydrophilic polymers of natural origin, including starch, pectin and alginates. WO 2014/128679 describes cosmetic compositions comprising at least one polymeric thickening agent of natural origin, including native or chemically modified starch, for example starch bound to acrylic polymers.

The said thickening agents of renewable origin, although preferable from the environmental point of view, have not however yet been able to effectively replace thickening agents of synthetic origin because their performance is not fully comparable to that of the latter and production costs are still not yet effectively competitive.

There is therefore a need to identify new thickening agents of renewable origin and biodegradable, which are capable of establishing themselves on the market as alternative thickening agents to synthetic ones.

Starting from this technical problem it has now surprisingly been found that it is possible to overcome the above mentioned problems, and in particular to obtain performance during use which is wholly similar to that which can be achieved using synthetic thickening agents at costs which, if not lower, are at least comparable to those of synthetic ones, by using destructurized starch as a thickening agent. This invention therefore relates to the use of destructurized starch as a thickening agent in a wide range of products, particularly cosmetic, dermatological and pharmaceutical compositions, paints, phytosanitary products and detergents.

The use of destructurized starch as a thickening agent unexpectedly imparts to the compositions containing it high stability to changes of pH and content of electrolytes (inter alia NaCl), thus widening the possibilities for application and ensuring better uniformity of performance regardless of conditions of use.

In the meaning of this invention, by destructurized starch is meant a starch of any type which has lost its native granular structure. As far as the native granular structure of starch is concerned, this can advantageously be identified by phase contrast optical microscopy. In one particularly preferred embodiment of this invention the destructurized starch is a starch which has completely lost its native granular structure, which is also known as “completely destructurized starch”.

Destructuring of the starch is advantageously carried out in any equipment capable of ensuring the temperature, pressure and shear force conditions suitable for destroying the native granular structure of the starch. Conditions suitable for obtaining complete destructuring of the starch are for example described in patents EP-0 118 240 and EP-0 327 505. Advantageously the starch is destructurized by means of an extrusion process at temperatures of between 110 and 250° ° C., preferably 130-180° C., preferably at pressures between 0.1 and 7 MPa, more preferably 0.3-6 MPa, and preferably providing a specific energy of more than 0.1 kWh/kg during the extrusion.

Destructuring of the starch preferably takes place in the presence of between 1 and 40% by weight, with respect to the weight of the starch, of one or more plasticizers selected from water and polyols having from 2 to 22 carbon atoms. As far as the water is concerned, this may also be that naturally present in the starch. Among the polyols, polyols having 1 to 20 hydroxyl groups containing 2 to 6 carbon atoms, their ethers, thioethers and organic and inorganic esters are preferred. Examples of polyols are glycerine, diglycerol, polyglycerol, pentaerythritol, polyglycerol ethoxylate, ethylene glycol, polyethylene glycol, 1,2-propanediol, 1,3-propanediol, 1,4-butanediol, neopentylglycol, sorbitol monoacetate, sorbitol diacetate, sorbitol monoethoxylate, sorbitol diethoxylate, and mixtures thereof. In a preferred embodiment the starch is destructurized in the presence of glycerol or a mixture of plasticizers comprising glycerol, even more preferably between 2 and 90% by weight of glycerol with respect to the total weight of plasticizers. Preferably the destructurized starch according to this invention comprises from 1 to 40% by weight of plasticizers with respect to the weight of the starch. A starch which can be used for the preparation of destructurized starch according to this invention is preferably selected from native starch (such as maize starch, potato starch, rice starch, tapioca starch), oxidized starch, dextrinized starch, etherified starch (such as preferably starch ethoxylate, silyl ethers of starch), starch esters (such as preferably starch hydroxypropylate, starch acetate), and mixtures thereof. Preferably the starch used for preparation of the destructurized starch is native starch.

In an embodiment thereof the present invention relates to the use of one or more “silyl ethers of destructurized starch” as a thickening agent, by this term being meant destructurized starches in which at least one oxygen atom in the destructurized starch is covalently bound to at least one silicon atom and/or at least one compound containing silicon.

As far as the compounds containing silicon are concerned, these are preferably selected from the group comprising organosilanes (including organodisilanes, organotrisilanes, organopolysilanes), halosilanes (including di-, tri- and polyhalosilanes), silanols (including di-, tri- and polysilanols), silazanes (including di-, tri- and polysilazanes). More preferably the silicon-containing compounds are selected from the organosilanes, even more preferably from those having a general formula selected from:

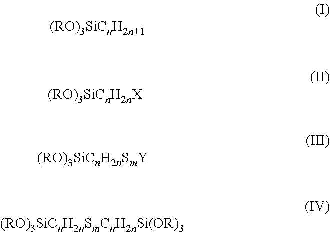

in which R represents an alkyl group having from 1 to 4 carbon atoms, the R being the same or different from each other;
“n” represents a whole number from 1 to 6;
“m” represents a whole number from 1 to 6;
X represents a mercaptan group, an amine group, a vinyl group, a nitroso group, an imide group, a phenyl group, a chlorine atom or an epoxy group;
Y represents a cyano group, an N,N-dimethyl thiocarbamoyl group, a mercaptobenzotriazole group, or a methacrylate group.

Organosilanes which contain no sulfur are particularly preferred.

As far as the silyl ethers of destructurized starch are concerned, these can be obtained by means of a single stage process or in several stages. In a first preferred embodiment the silyl ethers of destructurized starch are obtained by subjecting one or more silyl ethers of starch to temperature, pressure and shear force conditions suitable for destroying the native granular structure of the starch, in accordance with the teachings described above in respect of the destructuring process.

Alternatively, the silyl ethers of destructurized starch can preferably be obtained by mixing previously destructurized starch with at least one compound containing silicon at temperatures between 110 and 250° C., preferably 130-180° C. Mixing may take place in any equipment suitable for the purpose, preferably in a static mixer or extruder, more preferably in an extruder. During preparation of the silyl ethers of destructurized starch according to this invention the compounds containing silicon can be metered in excess with respect to the starch or in any event not caused to react completely with the latter, so that the silyl ethers of destructurized starch according to this invention may advantageously contain between 1 and 20% by weight of at least one silicon-containing compound, preferably organosilanes, halosilanes, silanols or silazanes which are not bound to an oxygen atom of the starch. More preferably the said silicon compound which is not bound to an oxygen atom of the starch is an organosilane.

Depending upon the type of products in which it is intended to be used as a thickening agent, it is also possible to add polymers containing hydrophilic groups intercalated with hydrophobic sequences, cross-linking agents, depolymerising agents and mixtures thereof with the destructurized starch.

As far as the polymers containing hydrophilic groups intercalated with hydrophobic sequences are concerned, these are preferably added to the destructurized starch in quantities of between 0.1 and 80% by weight with respect to the total composition of the thickening agent comprising the destructurized starch.

The said polymers containing hydrophilic groups intercalated with hydrophobic sequences are advantageously selected from:
A. polyvinyl alcohol having a degree of hydrolysis of between 10 and 100%;
B. vinyl alcohol/vinyl acetate block copolymers;
C. polyvinyl acetate, both in the dry form and in the form of aqueous emulsion;
D. copolymers of ethylene with vinyl alcohol, vinyl acetate, acrylic acid, methacrylic acid, crotonic acid, itaconic acid, maleic anhydride, glycidyl methacrylate or mixtures thereof;
E. 6-6, 6-9 or 12 aliphatic polyamides, aliphatic polyurethanes, aliphatic and aliphatic/aromatic polyesters, polyurethane/polyamide, polyurethane/polyether, polyurethane/polyester, polyamide/polyester, polyamide/polyether, polyester/polyether, polyurea/polyester, polyurea/polyether, polylactic acid, polyglycolic acid, polycaprolactone/urethane random or block copolymers, in which the molecular weight of the polycaprolactone blocks is between 300 and 3000.

Mixtures of the said polymers may also be used.

Among the polymers containing hydrophilic groups intercalated with hydrophobic sequences, those preferred are polyvinyl alcohols having a degree of hydrolysis between 10 and 100%, polyvinyl acetates in the dry form or in the form emulsified with water, vinyl alcohol/vinyl acetate block copolymers and mixtures thereof.

Among these polyvinyl alcohols having a degree of hydrolysis between 10 and 100% are particularly preferred.

In the case of copolymers of ethylene with vinyl alcohol, these preferably contain 20-50% in moles of ethylene units.

In the case of copolymers of ethylene with acrylic acid, these preferably contain 70-99% by weight of ethylene units.

The said polymers containing hydrophilic groups intercalated with hydrophobic sequences are added to the destructurized starch in any manner known to those skilled in the art.

The addition of the said polymers containing hydrophilic groups intercalated with hydrophobic sequences may be brought about at the same time as the starch is destructurized, or in a subsequent stage. A first method therefore comprises preparation in a single stage: according to this method the starch is destructurized and at the same time mixed with the said polymers containing hydrophilic groups intercalated with hydrophobic sequences. Alternatively the starch is first destructurized and then mixed with the said polymers containing hydrophilic groups intercalated with hydrophobic sequences.

Starch destructurized according to this invention is biodegradable when composted in the meaning of Standard EN13432.

Preferably, according to the present invention, destructurized starch is used as a thickening agent in cosmetic, dermatological and pharmaceutical compositions, paints, phytosanitary products and detergents. In a preferred embodiment thereof, the present invention relates to the use of destructurized starch as a thickening agent in cosmetic compositions.

The cosmetic, dermatological and pharmaceutical compositions, paints, phytosanitary products and detergents preferably comprise from 1 to 30% by weight, more preferably from 3 to 20% by weight, even more preferably from 5 to 15% by weight of destructurized starch as a thickening agent according to this invention, said percentages relating to the total weight of the respective compositions.

Preferably, thanks to the use of destructurized starch as a thickening agent, the cosmetic, dermatological and pharmaceutical compositions, paints, phytosanitary products and detergents have viscosity values in the range from 10 to 2000 Pa·s, preferably from 50 to 1500 Pa·s, more preferably 100-1000 Pa·s measured by means of rotational rheometer with a plate-plate geometry with a constant shear rate (time sweep test) and stress threshold values (Bingham behaviour) in the range 1-90 Pa, preferably in the range 10-80 Pa, more preferably 25-70 Pa using a plate-plate geometry rotational rheometer with a decreasing stress rate (stress rate test). Even more preferably, the destructurized starch is used in cosmetic, dermatological and pharmaceutical compositions, paints, phytosanitary products and detergents for modifying the viscosity at values in the range from 10 to 2000 Pa·s, preferably from 50 to 1500 Pa·s, more preferably 100-1000 Pa·s, and/or the stress threshold at values in the range 1-90 Pa, preferably in the range 10-80 Pa, more preferably in the range 25-70 Pa.

Depending upon their specific use, the cosmetic, dermatological and pharmaceutical compositions, paints, phytosanitary products and detergents have suitable pH values, preferably between 1 and 12.

Preferably, thanks to the use of destructurized starch as a thickening agent, the cosmetic, dermatological and pharmaceutical compositions, paints, phytosanitary products and detergents have viscosity of at least 100 Pa·s and stress threshold of at least of 1 Pa in the presence of electrolytes (such as NaCl).

The cosmetic, dermatological and pharmaceutical compositions, paints, phytosanitary products and detergents comprising destructurized starch as a thickening agent preferably take the form of fluids, gels, foams, sprays, lotions or creams. The cosmetic, dermatological and pharmaceutical compositions, paints, phytosanitary products and detergents are preferably formulated in the form of aqueous or lipophilic compositions, such as emulsions, solutions or dispersions.

The cosmetic, dermatological and pharmaceutical compositions, paints, phytosanitary products and detergents may also contain other additives and auxiliaries normally used in their corresponding fields of application, for example oils, waxes, surfactants, suspensory agents, preservatives, emulsifiers, co-emulsifiers, dispersants, surfactants, polymers, anti-foaming agents, solubilising agents, stabilisers, film-forming agents, other thickening agents, gelling agents, emollients, disinfectants, active ingredients, astringents, deodorants, sun filters, antioxidants, oxidants, humectants, solvents, pigments, colouring agents, texturing agents, perfumes, opacifiers and/or silicones. In particular phytosanitary products comprise one or more phytopharmaceuticals.

The invention will now be described with reference to some Examples, which are intended to be illustrative in purpose and not limiting upon the invention.

### Methods Used for Characterisation

### Viscosity and Bingham Behaviour Measurements

Viscosity measurements were carried out using TA Instruments ARES G-2 controlled strain rotational rheometer under steady state conditions with a plate-plate geometry.

In the case of measurements relating to the viscosities of the products the operating conditions were:
Ambient temperature (ca. 25° C.);
Gap (mm)=1;
Shear rate (1/s)=0.1;
Test time(s)=600.

In the case of measurements relating to Bingham behaviour (stress threshold) the operating conditions were:
Ambient temperature (ca. 25° C.);
Gap (mm)=1;
Stress rate (Pa)=100−50−1;
Test time(s)=360.

### Phase Contrast Microscopy

Phase contrast optical microscopy was carried out using a Leitz Wetzlar Orthoplan optical microscope at a magnification (Polaroid 545) of 400× with a Phaco 2 EF 40/0.65 objective lens, a no. 5 (phase contrast) polarising filter or no. H (polarised light) filter. Approximately 20 mg of sample were placed on an optical microscope slide together with a drop of distilled water. Using a spatula the sample was homogenised with the water until a slightly viscous paste was obtained. A spatula tip of that paste was then placed between two optical microscope slides and gently smeared so as to obtain a semi-transparent film, which was subsequently analysed.

### EXAMPLE 1—PREPARATION OF DESTRUCTURIZED STARCH FROM NATIVE MAIZE STARCH

A mixture comprising 75 parts by weight of Cargill C*Gel 03401 (12% water) native maize starch and 25 parts of water was fed to a TSA EM 21-40 co-rotating twin-screw extruder (diameter=21 mm, L/D=40) operating under the following conditions:
rpm (min-1)=100;
thermal profile (° C.): 60-80-140-170-160-140-120-110;
throughput (kg/h): 1.5;
degassing: closed;
head temperature (° C.): 110;
head pressure (bar): 5-6.

The destructurized starch so obtained was analysed using phase contrast optical microscopy and this showed that structures which could be attributed to the native granular structure of starch were completely absent.

### EXAMPLE 2—PREPARATION OF DESTRUCTURIZED STARCH FROM STARCH HYDROXYPROPYLATE

A mixture comprising 75 parts by weight of Ingredion Beneform 3750 starch hydroxypropylate (13% of water) and 25 parts of water was fed to a TSA EM 21-40 co-rotating twin-screw extruder (diameter=21 mm, L/D=40) operating under the following conditions:
rpm (min-1)=100;
thermal profile (° C.): 60-80-140-170-160-140-110-90;
throughput (kg/h): 2.2;
degassing: closed;
head temperature (° C.): 90;
head pressure (bar): 7-25.

The destructurized starch so obtained was analysed by phase contrast optical microscopy and showed that structures which could be attributed to the native granular structure of starch were completely absent.

### EXAMPLE 3—PREPARATION OF STARCH DESTRUCTURIZED FROM NATIVE MAIZE STARCH

A mixture comprising 75 parts by weight of Cargill C*Gel 03401 (12% water) native maize starch, 15 parts of water and 10 parts of glycerol was fed to a TSA EM 21-40 co-rotating twin-screw extruder (diameter=21 mm, L/D=40) operating under the following conditions:
rpm (min-1)=100;
thermal profile (C): 60-80-140-170-160-140-120-110;
throughput (kg/h): 1.5;
degassing: closed;
head temperature (° C.): 110;
head pressure (bar): 5-6.

The destructurized starch so obtained was analysed using phase contrast optical microscopy and this showed that structures which could be attributed to the native granular structure of starch were completely absent.

### EXAMPLES 4-7 AND 8 (COMPARATIVE)—PREPARATION OF COSMETIC COMPOSITIONS

Five cosmetic compositions were prepared using the destructurized starch prepared in Examples 1 and 2 as thickening agent, and as a comparison, native potato starch (compositions in Table 1).

TABLE 1

| | Composition (g) | | | | |
|---|---|---|---|---|---|
| | Example | Example | Example | Example | Example 8 |
| Component | 4 | 5 | 6 | 7 | (comparative) |
| Plantasens HP30 natural | 3 | 3 | 3 | 3 | 3 |
| emulsifier (Clariant) | | | | | |
| Myritol 318 softener (Clariant) | 12 | 12 | 12 | 12 | 12 |
| Aqueous solution of glycerine | 3 | 3 | 3 | 3 | 3 |
| (85% wt) | | | | | |
| Phenonip ME preservative | 1 | 1 | 1 | 1 | 1 |
| (Clariant) | | | | | |
| Destructurized starch according | 11 | 12 | — | — | — |
| to Example 1 | | | | | |
| Destructurized starch according | — | — | 7 | 10 | — |
| to Example 2 | | | | | |
| Maize native starch (Cargill) | — | — | — | — | 11 |
| Water | 70 | 69 | 74 | 71 | 70 |

Said compositions were prepared using the following preparation protocol:
A. Plantasens HP30 natural emulsifier (Clariant) and Myritol 318 softener (Clariant) were mixed in a 50 cc flask and heated to approximately 80° C. until they melted/dissolved and were set aside;
B. Then an aqueous solution of 85% by weight glycerine and water were mixed at 25° C. and these too were set aside;
C. The quantities of thickening agent shown in Table 1 were added to the mixture prepared in paragraph A in a polyethylene flask, stirring provided by an Ika Ultra-Turrax T 25 mixer set at 400 rpm;
D. The mixture containing the thickening agent obtained in paragraph C was then added to the mixture obtained in paragraph B with stirring provided by an Ika Ultra-Turrax T 25 (set at 13000 rpm), then keeping the whole stirred for 2 minutes. Subsequently stirring was reduced to 400 rpm, and the mixture so obtained was allowed to cool to 40° C.
E. When the mixture in paragraph D had reached a temperature below 40° C., Phenonip ME preservative (Clariant) was added, again with stirring (400 rpm), and the whole was allowed to cool to 25° C.
F. The pH was then measured and if outside the range 6.0-6.5, citric acid or NaOH was added as necessary in order to bring it within this range.
G. The composition so obtained was allowed to rest for 24 hours and then characterised by determining its viscosity and stress threshold (Table 2).

TABLE 2

| Example | Viscosity (Pa · s) | Stress Threshold(Pa) |
|---|---|---|
| 4 | 506 | 31 |
| 5 | 631 | 36 |
| 6 | 282 | 13 |
| 7 | 567 | 14 |
| 8 (comparative) | 40 | nd |

wherein, “nd” stands for not determinable.

### EXAMPLES 9, 10 AND 11 (COMPARATIVE)—ASSESSMENT OF THE EFFECT OF ELECTROLYTES CONTENT ON THE PERFORMANCE OF THICKENING AGENTS

In order to test the ability of the thickening agents according to the invention to maintain their properties as the electrolytes content changed, some cosmetic compositions were prepared following the protocol shown in Examples 4-7 and 8 (comparative) above, also adding 3 g of NaCl in paragraph B. Table 3 shows the quantities of thickening agent used to prepare the compositions and the viscosity and stress threshold values obtained.

TABLE 3

| | | | | Stress |
|---|---|---|---|---|
| | | Grams | Viscosity | Threshold |
| Example | Thickening agent | added | (Pa · s) | (Pa) |
| 9 | Destructurized | 11 | 356 | 13 |
| | starch according | | | |
| | to Example 1 | | | |
| 10 | Destructurized | 7 | 153 | 1 |
| | starch according | | | |
| | to Example 2 | | | |
| 11 (com- | Maize native | 11 | 25 | nd |
| parative) | starch | | | |

wherein, “nd” stands for not determinable.

Despite the addition of the electrolytes, the compositions according to Examples 9 and 10 appeared to maintain good performance, as shown by the small change in the viscosity and stress threshold values in comparison with Examples 4 and 6.

### EXAMPLES 12-17—ASSESSMENT OF THE EFFECT OF PH AND THE PERFORMANCE OF THICKENING AGENTS

In order to test the ability of thickening agents according to the invention to maintain their properties as the pH of the compositions varied, some cosmetic compositions were prepared following the protocol shown above in Examples 4-7 and 8 (comparative), modifying the pH of the compositions to values of 1, 6-6.5 and 12 in paragraph F through the addition of HCl, citric acid or NaOH as required. Table 4 shows the quantities of thickening agents used to prepare the compositions and the viscosity and stress threshold values obtained.

TABLE 4

| | | | | | Stress |
|---|---|---|---|---|---|
| | | Grams | | Viscosity | Threshold |
| Example | Thickening agent | added | pH | (Pa · s) | (Pa) |
| 12 | Destructurized | 11 | 1 | 744 | 65 |
| | starch according | | | | |
| | to Example 1 | | | | |
| 4 | Destructurized | 11 | 6-6.5 | 506 | 31 |
| | starch according | | | | |
| | to Example 1 | | | | |
| 13 | Destructurized | 11 | 12 | 718 | 38 |
| | starch according | | | | |
| | to Example 1 | | | | |
| 14 | Destructurized | 7 | 1 | 343 | 15 |
| | starch according | | | | |
| | to Example 2 | | | | |
| 6 | Destructurized | 7 | 6-6.5 | 282 | 13 |
| | starch according | | | | |
| | to Example 2 | | | | |
| 15 | Destructurized | 7 | 12 | 418 | 27 |
| | starch according | | | | |
| | to Example 2 | | | | |
| 16 (com- | Maize native | 11 | 1 | 45 | nd |
| parative) | starch | | | | |
| 8 (com- | Maize native | 11 | 6-6.5 | 40 | nd |
| parative) | starch | | | | |
| 17 (com- | Maize native | 11 | 12 | 48 | nd |
| parative) | starch | | | | |

Despite the wide variation of pH, the compositions comprising destructurized starch according to the present invention appeared to maintain good performance, as shown by the small change in the viscosity and stress threshold values throughout the pH range investigated.

1. A method of thickening a cosmetic composition, a dermatological composition, a pharmaceutical composition, a paint, a phytosanitary product or a detergent, which comprises the addition in the cosmetic composition, the dermatological composition, the pharmaceutical composition, the paint, the phytosanitary product or the detergent atological composition, the pharmaceutical composition, the paint, the phytosanitary product or the detergent of a destructurized starch as a thickening agent, wherein said destructurized starch has completely lost its native granular structure and is obtained from a starch in the presence of from 1% to 40% by weight, with respect to the weight of said starch, of one or more plasticizers selected from water and polyols having from 2 to 22 carbon atoms, wherein the thickened composition has a viscosity from 50 to 2000 Pa·s, and wherein the destructurized starch does not comprise a thermoplastic polymer.

2. The method according to claim 1, in which the said destructurized starch is obtained from a starch selected from the group consisting of native starch, oxidized starch, dextrinized starch, starch ethers, starch esters and mixtures thereof.

3. A cosmetic composition, a dermatological composition, a pharmaceutical composition, a paint, a phytosanitary product or a detergent comprising destructurized starch as a thickening agent comprising from 1% to 30% by weight, with respect to the total weight of the cosmetic composition, the dermatological composition, the pharmaceutical composition, the paint, the phytosanitary product or the detergent, of said destructurized starch, wherein said destructurized starch has completely lost its native granular structure and is obtained from a starch in the presence of from 1% to 40% by weight, with respect to the weight of said starch, of one or more plasticizers selected from water and polyols having from 2 to 22 carbon atoms, wherein the thickened composition has a viscosity from 50 to 2000 Pa·s, and wherein the destructurized starch does not comprise a thermoplastic polymer.

4. The composition according to claim 3, in which the said destructurized starch is obtained from a starch selected from the group consisting of native starch, oxidized starch, dextrinized starch, starch ethers, starch esters, and mixtures thereof.

5. The composition according to claim 3, characterized by a viscosity within the range 10-2000 Pa·s measured by means of a plate-plate geometry rotational rheometer at constant shear rate (time sweep test) at 25° C.

6. The composition according to claim 3, characterized by a stress threshold value in the range 1-90 Pa using plate-plate geometry rotational rheometer with decreasing stress rate (stress rate test) at 25° C.

7. The composition according to claim 3, in the form selected from the group of a fluid, gel, foam, spray, lotion and cream.

8. The composition according to claim 3, having an aqueous or lipophilic nature.

9. The composition according to claim 8, in the form selected from the group of an emulsion, solution and dispersion.

10. The composition according to claim 4, characterized by a viscosity within the range 10-2000 Pa·s measured by means of a plate-plate geometry rotational rheometer at constant shear rate (time sweep test) at 25° C.

11. The composition according to claim 10, characterized by a stress threshold value in the range 1-90 Pa using plate-plate geometry rotational rheometer with decreasing stress rate (stress rate test) at 25° C.

12. The composition according to claim 4, characterized by a stress threshold value in the range 1-90 Pa using plate-plate geometry rotational rheometer with decreasing stress rate (stress rate test) at 25° C.

13. The composition according to claim 5, characterised by a stress threshold value in the range 1-90 Pa using plate-plate geometry rotational rheometer with decreasing stress rate (stress rate test) at 25° ° C.

14. The composition according to claim 4, in the form selected from the group of a fluid, gel, foam, spray, lotion and cream.

15. The composition according to claim 5, in the form selected from the group of a fluid, gel, foam, spray, lotion and cream.

16. The composition according to claim 6, in the form selected from the group of a fluid, gel, foam, spray, lotion and cream.

17. The composition according to claim 4, having an aqueous or lipophilic nature.

18. The composition according to claim 5, having an aqueous or lipophilic nature.

19. The composition according to claim 6, having an aqueous or lipophilic nature.

20. The composition according to claim 7, having an aqueous or lipophilic nature.
